(19) United States
(12) Patent Application Publication
(10) Pub. No.: US 2016/0199517 A1
(43) Pub. Date: Jul. 14, 2016

(54) MRI EVALUATION OF HETEROGENEOUS TISSUE

(71) Applicant: The Board of Trustees of the Leland Stanford Junior University, Stanford, CA (US)

(72) Inventors: Phillip C. Yang, Stanford, CA (US); Rajesh Dash, Stanford, CA (US)

(21) Appl. No.: 14/992,847

(22) Filed: Jan. 11, 2016

Related U.S. Application Data
(60) Provisional application No. 62/102,916, filed on Jan. 13, 2015.

(51) Int. Cl. A61K 49/10

(52) U.S. Cl. CPC A61K 49/10

(57) ABSTRACT

The methods of the invention exploit the capabilities of manganese-enhanced MRI (MEMRI), which provides viability-specific biological contrast agent through the intracellular accumulation of Mn²⁺. A typical contrast agent utilizes non-chelated Mn²⁺ in combination with Ca²⁺, e.g. as calcium gluconate. Active intracellular accumulation of Mn²⁺ generates high signal from the viable cells in normal tissue, or normal regions of heterogeneous tissue; no signal from the non-viable cells; and intermediate signal from viable but injured cells. The intermediate signal defines a “gray zone” of potentially salvageable cells.

### BACKGROUND OF THE INVENTION

Ischemic cardiomyopathy (ICM) is the primary etiology of advanced heart failure (HF), the leading diagnosis of hospital admissions in the US. Clinical studies have confirmed that the high morbidity and mortality of HF are associated with ventricular arrhythmias and LV remodeling in the peri-infarct region (PIR). Indeed, patients with a history of acute MI and LV dysfunction have a 6 month mortality >10%, one third of which is attributed to sudden cardiac death. The critical role of tissue heterogeneity in the PIR, independent of actual infarct size, has been recognized as an important substrate to trigger these ventricular arrhythmias. To this end, preclinical studies have confirmed that the heterogeneous PIR contains the critical isthmus for ventricular tachycardia and successful therapy requires ablation of the isthmus.

Studies have also demonstrated that revascularization of the ischemic PIR results in a lower incidence of ventricular arrhythmias and LV dilatation. While revascularization may mitigate ventricular arrhythmias acutely, the Multicenter UnSustained Tachycardia Trial (MUSTT) noted that following a reperfused MI, patients were increasingly likely to have ventricular arrhythmias as their infarct matured. Therefore, an accurate tissue characterization of the PIR is critical in determining which patients will benefit from revascularization and/or medical therapy targeted at PIR.

In order to evaluate the role of the PIR, clinical studies have employed cardiac MRI (CMR) to assess PIR in patients with severe ICM, which confirmed that precise tissue characterization of the PIR predicts future cardiovascular events while traditional measures including scar presence, LVEF, and LV volumes did not demonstrate such significance. The trials revealed the need for a more sensitive method to detect salvageable myocardium within and around areas of cardiac injury to allow clinicians to guide therapy. Compositions and methods are provided for as a means of distinguishing through imaging between necrosed cells, injured cells and fully viable cells in vivo, using manganese enhanced MRI.

U.S. Pat. No. 5,980,863 is directed to compositions for MRI imaging of tissue. Manganese-enhanced MRI (MEMRI) exploits the T1 shortening effect of Mn²⁺ to generate positive MRI contrast. Also see Chung et al. (2012) Magn Reson Med. 68(2):595-9; and Dash et al. (2011) Circ Cardiovasc Imaging. 4(5):574-82.

### SUMMARY OF THE INVENTION

Compositions and methods are provided for MRI analysis in vivo of heterogeneous tissue, in which small numbers of viable cells may be present within tissue comprising non-viable cells. In some embodiments the heterogeneous tissue is adjacent to a region of necrosis, for example an infarcted region.

The methods of the invention exploit the capabilities of manganese-enhanced MRI (MEMRI), which provides viability-specific biological contrast agent through the intracellular accumulation of Mn²⁺. A contrast agent for use in the methods of the invention may utilize non-chelated Mn²⁺ in combination with Ca²⁺, e.g. as calcium gluconate. Active intracellular accumulation of Mn²⁺ generates high signal from the viable cells in normal tissue, or normal regions of heterogeneous tissue; no signal from the non-viable cells; and intermediate signal from viable but injured cells. The intermediate signal defines a “gray zone” of potentially salvageable cells.

In some embodiments of the invention, the MRI analysis of heterogeneous tissue is performed over time, where an individual is monitored for at least two time points, and may be monitored over a series of time points. In some embodiments, therapeutic intervention, which may be a part of a clinical trial, is performed during the period of time between analysis. therapeutic intervention may include, without limitation, cell therapies, e.g. introduction of regenerative cells to the tissue; biological therapies, such as gene therapy to introduce regenerative genetic and/or proteins to the injured cells; contacting the cells with antibodies, cytokines, growth factors, and the like to modulate cell growth and/or regeneration; drug therapies, e.g. treating the patient with a pharmaceutical agent to modulate the intermediate zone cells; revascularization and reperfusion treatments, e.g. stents, surgical bypass and other revascularization modalities; environmental strategies; diet intervention; and other modes of therapy that may affect the growth and biological activity of the gray zone cells.

In some embodiments of the invention, a combination approach the differing capabilities of MEMRI and delayed-enhanced MRI (DEMRI), which distributes primarily within the extracellular space to generate a bright MRI signal in infarcted tissue; to enhance the identification and visualization of the gray zone cells. The combined use of these methods may be referred to herein as dual contrast MRI. The MEMRI and DEMRI analysis may be performed substantially concomitantly, or may be performed sequentially, alternately, etc.

In some embodiments the tissue being imaged is damaged cardiac tissue. In some such embodiments the tissue being imaged is infarcted cardiac tissue, e.g. in a patient following an episode of myocardial ischemia, and particularly in patients having or suspected of having myocardial infarction, which may be transmural or non-transmural. In such embodiments, the imaging identifies a bright signal from non-infarcted tissue, no signal from infarcted tissue, and an intermediate, gray zone signal from viable cells within the peri-infarct region. The resulting pattern allows high-resolution, steady-state imaging of myocardial viability and injury. Cells providing an intermediate signal are of particular interest for analysis during therapeutic strategies, because these cells have potential for salvage and restoration of function, but have also been identified as a source of ventricular arrhythmias.

In another embodiment, the cardiac tissue is atrial wall tissue, e.g. following atrial wall damage. Atrial wall damage can be included in assessment of myocardial infarction, but may be assessed for other conditions, including without limitation: aneurysm; conditions relating to atrial arrhythmia; left atrial (LA) wall injury after pulmonary vein antrum isolation (PVAI) in patients with atrial fibrillation (AF); left atrial (LA) substrate remodeling (SRM) in patients with rheumatic mitral valve disease and persistent atrial fibrillation (AF); following coronary artery bypass grafting (CABG); and the like. In such embodiments, the imaging identifies a bright signal from normal tissue, no signal from non-viable tissue, and an intermediate, gray zone signal from viable cells within the damaged region. The resulting pattern allows high-resolution, steady-state imaging of atrial wall viability and injury.

### BRIEF DESCRIPTION OF THE DRAWINGS

FIG. 1 The percentage Peri-Infarct Region (PIR) per total LV volume.

FIG. 2 The percentage Peri-Infarct Region (PIR) per DEMRI enhanced volume.

### DETAILED DESCRIPTION OF THE EMBODIMENTS

Although preferred embodiments of the invention are described below, it should be understood that the present disclosure is to be considered as an exemplification of the principles of this invention and is not intended to limit the invention to the embodiments illustrated.

The methods of the invention provide a means of distinguishing through imaging between injured cells and fully viable cells in vivo, using manganese enhanced MRI. The application of a manganese imaging contrast agent to a living tissue of interest allows distinction based on the uptake of the contrast agent, which differs between dead or necrotic cells, normal viable cells, and injured cells.

Heterogeneous tissue comprises cells that are heterogeneous with respect to viability, for example comprising dead, substantially normal, and injured cells. The distribution and the level of injury is often variable as well. It will be understood by one of skill in the art that there is a continuous range of physiologic activity, and that the guidelines provided herein are intended to encompass that natural biological variability.

The physiology of a cell may be characterized with reference to a variety of parameters that are associated with normal cell function, including without limitation utilization of substrates, for example glucose, O₂, and the like; with reference to pathway activity, including without limitation glycolysis, citric acid cycle, oxidative phosphorylation, etc.; with reference to replicative ability if applicable, i.e. if a cell is normally dividing; with respect to the normal function of the cell, for example contractility, synthesis of hormones, excitation, and the like; with respect to the normal life span of the normal cell; or any other suitable measure of viability.

The term viable cells refers to those cells having the cellular anatomy and/or physiologic activity of the cells normally resident in the tissue of interest. For example a potentially injured neural cell may be compared to a normal neural cell, a cardiomyocyte to a normal cardiomyocyte, and the like. Generally, a cell considered to be viable will have at least 50% of the normal parameters of the cognate cell type, at least about 75% of the normal parameters, at least about 80% of the normal parameters, at least about 85% of the normal parameters, at least about 90% of the normal parameters, at least about 95% of the normal parameters, at least about 98%, at least about 99%, and may be 100% or more.

Injured cells as defined herein have one or more reduced parameters associated with normal cell function, as described above, and may have, for example, up to about 10%, up to about 20%, up to about 30%, up to about 40%, up to about 50%, up to about 60%, up to about 70%, up to about 80%, up to about 90%, up to about 95%, and less than about 100%.

There are many causes of cell injury, including without limitation hypoxia, ischemia, the presence of physical changes such as temperature, trauma, radiation; the presence of chemical agents such as toxins, drugs; etc.; the presence of infection; the presence of inflammatory or other immune responses; nutritional imbalance; genetic changes; and the like.

The methods of the present invention may be benchmarked with respect to structural characteristics of injured cells, which may include without limitation cell swelling, detachment of ribosomes from granular e.r., dissociation of polysomes into monosomes; pallor, hydropic change, vacuolar degeneration, plasma membrane blebbing, blunting, villous distortion, myelin figures, mitochondrial swelling, rarefaction, nuclear disaggregation of granular and fibrillar elements.

Other examples of structural features useful in benchmarking cell injuries include, without limitation, a scoring system based on ultrastructure. In one example for analysis of cardiac tissue, each identified cardiomyocyte may be graded on whether it exhibited a high abundance (5), moderate abundance (4), low abundance (3), rare (2), or complete absence (1) of a feature. Conversely, for an unhealthy tissue feature, each identified cardiomyocyte may be graded with 5 indicating that the nucleus displayed complete absence, 4 indicating rare, 3 indicating low abundance, 2 indicating moderate abundance, and 1 indicating high abundance of the unhealthy feature. For example, the complete absence of a healthy feature or the high abundance of an unhealthy feature yields a score of 1. Features include in the nucleus: notched/furrowed membrane; homogenous chromatin granules; chromatin accumulated along nuclear membrane; chromatin clots within nucleus; dense chromatin; dark chromatin finely structured. Features in mitochondria include dense perinuclear accumulation; fine filaments/glycogen granules between nucleus and mitochondria; destroyed cristae; few mitochondria near nucleus; mitochondria isolated in niche. Features in myofibrils include myofibrils aligned in 1 row; t-tubules contain basal lamina; myofibrils are contracted; z-line disruption; lipid droplets between ruptured myofibrils.

The difference in staining between viable and injured cells provides a useful guide for therapy, in that tissue containing injured but not dead or infarcted cells may be amenable to treatment. Staining densities may be compiled into a signature pattern that is useful for prognosis and for theranostic purposes.

Generally with respect to an infarct, or other region of scarring or region of necrosis, a remote tissue is considered to be viable or substantially normal, the infarct or other damaged region can contain non-viable cells, and the “border” are cells that are injured but may be salvaged with therapy. The difference in intensity of staining is from about 1.25-fold, about 1.5-fold, about 1.75-fold, about 2-fold, about 2.25 fold, about 2.5 fold or greater decreased between the remote (viable) cells and injured cells. The difference in intensity of staining is from about 3-fold, about 3.5-fold, about 4-fold, about 4.5-fold, about 5-fold or greater decreased between the remote (viable) cells and dead cells in the infarct. The difference in intensity of staining is from about 2.5-fold, about 2.75-fold, about 3-fold, about 3.25-fold, about 3.5-fold, about 3.75-fold, about 4-fold, about 4.25-fold or greater increased between the injured (or gray zone) cells and dead cells in the non-viable region.

The signature pattern may be generated from imaging data as described above. The readout may be a mean, average, median or the variance or other statistically or mathematically-derived value associated with the measurement. Following obtainment of the signature pattern from the sample being assayed, the signature pattern can be compared with a reference or control profile to make a prognosis regarding the phenotype of the tissue from which the sample was obtained/derived. Typically a comparison is made with a sample or set of samples from an unaffected, normal source, from a known diagnosis, etc. An algorithm that compiles the imaging results will discriminate between individuals in different classifications with respect to prognosis for response to therapy.

An analytic classification process may use any one of a variety of statistical analytic methods to manipulate the quantitative data and provide for classification of the sample. Examples of useful methods include linear discriminant analysis, recursive feature elimination, a prediction analysis of microarray, a logistic regression, a CART algorithm, a FlexTree algorithm, a LART algorithm, a random forest algorithm, a MART algorithm, machine learning algorithms; etc. Classification can be made according to predictive modeling methods that set a threshold for determining the probability that a sample belongs to a given class. The probability preferably is at least 50%, or at least 60% or at least 70% or at least 80% or higher. Classifications also may be made by determining whether a comparison between an obtained dataset and a reference dataset yields a statistically significant difference. If so, then the sample from which the dataset was obtained is classified as not belonging to the reference dataset class. Conversely, if such a comparison is not statistically significantly different from the reference dataset, then the sample from which the dataset was obtained is classified as belonging to the reference dataset class.

The predictive ability of a model may be evaluated according to its ability to provide a quality metric, e.g. AUC or accuracy, of a particular value, or range of values. In some embodiments, a desired quality threshold is a predictive model that will classify a sample with an accuracy of at least about 0.7, at least about 0.75, at least about 0.8, at least about 0.85, at least about 0.9, at least about 0.95, or higher. As an alternative measure, a desired quality threshold may refer to a predictive model that will classify a sample with an AUC (area under the curve) of at least about 0.7, at least about 0.75, at least about 0.8, at least about 0.85, at least about 0.9, or higher. As is known in the art, the relative sensitivity and specificity of a predictive model can be “tuned” to favor either the selectivity metric or the sensitivity metric, where the two metrics have an inverse relationship. The limits in a model as described above can be adjusted to provide a selected sensitivity or specificity level, depending on the particular requirements of the test being performed. One or both of sensitivity and specificity may be at least about at least about 0.7, at least about 0.75, at least about 0.8, at least about 0.85, at least about 0.9, or higher.

### Cardiac Indication and Uses

In some embodiments of the invention, the tissue being analyzed is heart tissue. The heart needs to be supplied with a sufficient quantity of oxygen to prevent underperfusion. When reduced perfusion pressure distal to stenoses is not compensated by autoregulatory dilation of the resistance vessels, ischemia, meaning a lack of blood supply and oxygen, occurs. Because the zone least supplied generally is the farthest out, ischemia generally appears in areas farthest away from the blood supply.

After total or near-total occlusion of a coronary artery, myocardial perfusion occurs by way of collaterals, meaning vascular channels that interconnect epicardial arteries. Collateral channels may form acutely or may preexist in an under-developed state before the appearance of coronary artery disease. Preexisting collaterals are thin-walled structures ranging in diameter from 20 μm to 200 μm, with a variable density among different species. Preexisting collaterals normally are closed and nonfunctional, because no pressure gradient exists to drive flow between the arteries they connect. After coronary occlusion, the distal pressure drops precipitously and pre-existing collaterals open virtually instantly.

The term “myocardial ischemia” refers to a decrease in blood supply and oxygen to the cells of the myocardium. The development of myocardial ischemia has been attributed to two mechanisms: (1) increased myocardial oxygen demand, and (2) decreased myocardial perfusion and oxygen delivery. Myocardial ischemia generally appears first and is more extensive in the subendocardial region, since these deeper myocardial layers are farthest from the blood supply, with greater need for oxygen.

Chronic Myocardial lschemia. The term “chronic myocardial ischemia (CMI)” as used herein refers to a prolonged subacute or chronic state of myocardial ischemia due to narrowing of a coronary blood vessel in which the myocardium “hibernates”, meaning that the myocardium downregulates or reduces its contractility, and hence its myocardial oxygen demand, to match reduced perfusion, thereby preserving cellular viability and evading apoptosis. The underlying mechanism by which the myocardium does so is poorly understood. This hibernating myocardium is capable of returning to normal or near-normal function on restoration of an adequate blood supply. Once coronary blood flow has been restored to normal or near normal and ischemia is resolved, however, the hibernating myocardium still does not contract. This flow-function mismatch resulting in a slow return of cardiac function after resolution of ischemia has been called stunning. The length of time for function to return is quite variable, ranging from days to months, and is dependent on a number of parameters, including the duration of the original ischemic insult, the severity of ischemia during the original insult, and the adequacy of the return of the arterial flow.

Acute Myocardial Infarction (AMI). Another type of insult occurs during AMI. AMI is an abrupt change in the lumen of a coronary blood vessel which results in ischemic infarction, meaning that it continues until heart muscle dies. On gross inspection, myocardial infarction can be divided into two major types: transmural infarcts, in which the myocardial necrosis involves the full or nearly full thickness of the ventricular wall, and subendocardial (nontransmural) infarcts, in which the myocardial necrosis involves the subendocardium, the intramural myocardium, or both, without extending all the way through the ventricular wall to the epicardium. There often is total occlusion of the vessel with ST segment elevation because of thrombus formation within the lumen as a result of plaque rupture. The prolonged ischemic insult results in apoptotic and necrotic cardiomyocyte cell death. Necrosis compromises the integrity of the sarcolemmal membrane and intracellular macromolecules such that serum cardiac markers, such as cardiac-specific troponins and enzymes, such as serum creatine kinase (CK), are released. In addition, the patient may have electrocardiogram (ECG) changes because of full thickness damage to the muscle.

Acute myocardial infarction remains common with a reported annual incidence of more than one million cases in the United States alone. Preclinical and clinical data demonstrate that following a myocardial infarction, the acute loss of myocardial muscle cells and the accompanying peri-infarct zone hypoperfusion result in a cascade of events causing an immediate diminution of cardiac function, with the potential for long term persistence. The extent of myocardial cell loss is dependent on the duration of coronary artery occlusion, existing collateral coronary circulation and the condition of the cardiac microvasculature. Because myocardial cells have virtually no ability to regenerate, myocardial infarction leads to permanent cardiac dysfunction due to contractile-muscle cell loss and replacement with nonfunctioning fibrotic scarring. Moreover, compensatory hypertrophy of viable cardiac muscle leads to microvascular insufficiency that results in further demise in cardiac function by causing myocardial muscle hibernation and apoptosis of hypertrophied myocytes in the peri-infarct zone.

Among survivors of myocardial infarction, residual cardiac function is influenced by the extent of ventricular remodeling. The term ventricular remodeling refers to alteration in ventricular architecture, with associated increased volume and altered chamber configuration, driven on a histologic level by a combination of pathologic myocyte hypertrophy, myocyte apoptosis, myofibroblast proliferation, and interstitial fibrosis. Although originally described after myocardial infarction (MI), ventricular remodeling develops in response to a variety of forms of myocardial injury and increased wall stress.

Alterations in ventricular topography (meaning the shape, configuration, or morphology of a ventricle) occur in both infarcted and healthy cardiac tissue after myocardial infarction. Ventricular dilatation (meaning a stretching, enlarging or spreading out of the ventricle) causes a decrease in global cardiac function and is affected by the infarct size, infarct healing and ventricular wall stresses. Recent efforts to minimize remodeling have been successful by limiting infarct size through rapid reperfusion (meaning restoration of blood flow) using thromobolytic agents and mechanical interventions, including, but not limited to, placement of a stent, along with reducing ventricular wall stresses by judicious use of pre-load therapies and proper after-load management. Regardless of these interventions, a substantial percentage of patients experience clinically relevant and long-term cardiac dysfunction after myocardial infarction. Despite revascularization of the infarct related artery circulation and appropriate medical management to minimize ventricular wall stresses, a significant percentage of these patients experience ventricular remodeling, permanent cardiac dysfunction, and progressive deterioration of cardiac function, and consequently remain at an increased lifetime risk of experiencing adverse cardiac events, including death.

At the cellular level, immediately following a myocardial infarction, transient generalized cardiac dysfunction uniformly occurs. In the setting of a brief (i.e., lasting three minutes to five minutes) coronary artery occlusion, energy metabolism is impaired, leading to demonstrable cardiac muscle dysfunction that can persist for up to 48 hours despite immediate reperfusion. Coronary artery occlusion of more significant duration, i.e., lasting more than five minutes, leads to myocardial ischemia and is associated with a significant inflammatory response that begins immediately after reperfusion and can last for up to several weeks.

The Peri-Infarct Border Zone. The zone of dysfunctional myocardium produced by coronary artery occlusion extends beyond the infarct region to include a variable boundary of adjacent normal appearing tissue. This ischemic, but viable, peri-infarct zone of tissue separates the central zone of progressive necrosis from surrounding normal myocardium. The peri-infarct zone does not correlate with enzymatic parameters of infarct size and is substantially larger in small infarcts.

It is known that after an AMI, transient ischemia occurs in the border zones and that percutaneous coronary interventions, which open up the infarct-related artery, can adversely affect the health of the peri-infarct border zones. It has been suggested that intermediate levels of mean blood flow can exist as the result of admixture of peninsulas of ischemic tissue intermingled with regions of normally perfused myocardium at the border of an infarct. Progressive dysfunction of this peri-infarct myocardium over time may contribute to the transition from compensated remodeling to progressive heart failure after an AMI.

Atrial fibrillation or other types of atrial arrhythmia. (AF or A-fib) is an abnormal heart rhythm characterized by rapid and irregular beating. Heart-related risk factors include heart failure, coronary artery disease, cardiomyopathy, and congenital heart disease. Valvular heart disease often occurs as a result of rheumatic fever. Lung-related risk factors include COPD, obesity, and sleep apnea. AF is often treated with medications to achieve rate control or rhythm control.

AF is usually accompanied by symptoms related to a rapid heart rate. Rapid and irregular heart rates may be perceived as palpitations or exercise intolerance and occasionally may produce anginal chest pain (if the high heart rate causes ischemia). Other possible symptoms include congestive symptoms such as shortness of breath or swelling. The arrhythmia is sometimes only identified with the onset of a stroke or a transient ischemic attack (TIA). Some genetic conditions are associated with AF.

The primary pathologic change seen in atrial fibrillation is the progressive fibrosis of the atria. This fibrosis is due primarily to atrial dilation; however, genetic causes and inflammation may be factors in some individuals. Dilation of the atria can be due to almost any structural abnormality of the heart that can cause a rise in the pressure within the heart. This includes valvular heart disease (such as mitral stenosis, mitral regurgitation, and tricuspid regurgitation), hypertension, and congestive heart failure. Any inflammatory state that affects the heart can cause fibrosis of the atria. This is typically due to sarcoidosis but may also be due to autoimmune disorders that create autoantibodies against myosin heavy chains. Mutation of the lamin AC gene is also associated with fibrosis of the atria that can lead to atrial fibrillation.

Once dilation of the atria has occurred, this begins a chain of events that leads to the activation of the renin aldosterone angiotensin system (RAAS) and subsequent increase in matrix metalloproteinases and disintegrin, which leads to atrial remodeling and fibrosis, with loss of atrial muscle mass. Fibrosis is not limited to the muscle mass of the atria and may occur in the sinus node (SA node) and atrioventricular node (AV node), correlating with sick sinus syndrome.

Radiofrequency ablation to achieve PVAI is a promising approach to curing AF. Controlled lesion delivery and scar formation within the LA are indicators of procedural success, but the assessment of these factors has been limited to invasive methods. Noninvasive evaluation of LA wall injury to assess permanent tissue injury can be an important step in improving procedural success. The Maze procedure is an effective invasive surgical treatment that is designed to create electrical blocks or barriers in the atria of the heart, forcing electrical impulses that stimulate the heartbeat to travel down to the ventricles.

The term “cardiac biomarkers” refers to enzymes, proteins and hormones associated with heart function, damage or failure that are used for diagnostic and prognostic purposes. Different cardiac biomarkers have different times that their levels rise, peak, and fall within the body, allowing them to be used, not only to track the progress of a heart attack, but to estimate when it began and to monitor for recurrence. Some of the tests are specific for the heart while others also are elevated with skeletal muscle damage. Current cardiac biomarkers include, but are not limited to CK (creatine phosphokinase or creatine kinase) and CK-MB (creatine kinase-myoglobin levels (to help distinguish between skeletal and heart muscle)), troponin (blood levels of troponin I or T will remain high for 1-2 weeks after a heart attack; troponin generally is not affected by damage to other muscles), myoglobin (to determine whether muscle, particularly heart muscle, has been injured), and BNP (brain natriuretic peptide) or NT-proBNP (N-terminal prohormone brain natriuretic peptide (to help diagnose heart failure and grade the severity of that heart failure).

The term “cardiac catheterization” refers to a procedure in which a catheter is passed through an artery to the heart, and into a coronary artery. This procedure produces angiograms (i.e., x-ray images) of the coronary arteries and the left ventricle, the heart's main pumping chamber, which can be used to measure pressures in the pulmonary artery, and to monitor heart function.

The term “disease” or “disorder”, as used herein, refers to an impairment of health or a condition of abnormal functioning. The term “syndrome,” as used herein, refers to a pattern of symptoms indicative of some disease or condition. The term “condition”, as used herein, refers to a variety of health states and is meant to include disorders or diseases caused by any underlying mechanism or disorder, injury, and the promotion of healthy tissues and organs.

### Methods of Analysis

An individual suspected of having an infarct or other region of tissue necrosis, including without limitation fibrosis, scarring etc. associated with atrial fibrillation and treatment thereof, is analyzed by MEMRI for the presence of intermediate, or gray zone cells in regions surrounding the necrotic or fibrotic tissue. Any tissue may be analyzed. In certain such embodiments the infarct is a myocardial infarct, and the individual has been diagnosed with MI, or suspected of having MI. In other embodiments the tissue is atrial wall tissue.

The Mn contrast agent may be any agent that provides Mn ions safely to the patient. In some embodiments, the MEMRI contrast agent contains non-chelated Mn²⁺ (12%) with calcium gluconate (10%). Although not required, toxicity modifiers could be used with the contrast agent. The contrast agent may be administered intravenously as a bolus or as an infusion over a period of time. Commonly, though not necessarily, the infusion will be over a period of 1 minute to 30 minutes. Larger doses improve the imaging of organs, such as the heart, that take up manganese less efficiently than does liver. One may slow the rate of administration to increase the duration of signal intensity enhancement of blood without increasing total dose.

In a preferred embodiment, Mn gluconate/Ca gluconate (1:8), is parenterally administered over periods ranging from 10 seconds to 20 minutes. Dosing is related to target organ of interest and may range from from 1 μmol/kg body weight to 100 μmol/kg body weight of a source of Mn++ ion together with from 2 μmol/kg body weight to 1400 μmol/kg body weight of a source of Ca++ ions. Preferably the source of manganese is administered at 2 μmol/kg body weight to 30 μmol/kg body weight and the source of calcium is administered at 4 μmol/kg body weight to 400 μmol/kg body weight. Most preferably the source of manganese is administered at 3 μmol/kg body weight to 15 μmol/kg body weight and the source of calcium is administered at 6 μmol/kg body weight to 200 μmol/kg body weight. MRI is performed from during or immediately post dosing to 24 hours post dosing (vascular indications excepted). The rate of administration may be varied to further improve the cardiovascular tolerability of the contrast agent without an adverse effect on image quality, to increase the duration of the vascular phase of the agent, or to increase the dose without reducing the therapeutic index of the agent in order to enable imaging of target organs that accumulate manganese less efficiently than does liver.

Following administration of the contrast agent, MRI imaging is performed. MEMRI parameters, including T₁ and T₂ values, are measured. The data are analyzed to extract T₁ and T₂ values through nonlinear least-square fits to the inversion recovery and spin-echo decay curves, respectively. These unique in vivo properties allow the infarcted tissue to take up negligible amount, peri-infarct regions reduced amount, and normal tissue greatest amount for MEMRI enhancement. These properties enable precise determination of the baseline cardiac injury and subsequent restoration by delineating the direct changes in the regional viability at a cellular level.

By quantifying T1 values for each voxel in the myocardium, a parametric map can be generated representing the T1 relaxation times of any region of the heart without the need to compare it to a normal reference standard before or after the use of a contrast agent. Alternative implementation of T1-mapping useds variable sampling of the k-space in time (VAST), acquiring images in three to four breath-holds and correlating that data to invasive biopsy. Other sequences have been used for quantification of T1 as well using inversion recovery TrueFISP or multishot saturation recovery images. The most widely used T1-mapping sequence is based on the Modified LookLocker Inversion-recovery (MOLLI) technique. It consists of a single shot image with acquisitions over different inversion time readouts allowing for magnetization recovery of a few seconds after 3 to 5 readouts.

In some embodiments, dual contrast MEMRI-DEMRI analysis is performed. The novel dual contrast MEMRI-DEMRI can point to the viable cardiomyocytes within the PIR of transmural delayed enhancement. The discrepancy lies within the PIR that are positive for both MEMRI (viable) and DEMRI (non-viable) signal. The PIRs also display lower signal to noise ratio (SNR) by MEMRI than remote zones and lower SNR by DEMRI than core infarct zones, reflecting the heterogeneity of PIR with significant population of viable cardiomyocytes with intact Ca²⁺-channel function (MEMRI positive) in the PIR of the adjacent necrotic tissue (DEMRI positive).

The information thus obtained regarding the status of intermediate zone tissue is particularly relevant for analysis of patients undergoing therapy, and to assess the efficacy of clinical trials and treatment modalities in restoring function of the damaged tissue.

In some preferred embodiments, the methods of the invention are used in determining the efficacy of a therapy for treatment of a cardiovascular disease, either at an individual level, or in the analysis of a group of patients, e.g. in a clinical trial format. Such embodiments typically involve the comparison of two time points for a patient or group of patients. The patient status is expected to differ between the two time points as the result of a therapeutic agent, therapeutic regimen, or disease challenge to a patient undergoing treatment.

The terms “therapeutically effective”, “infarct area-improving amount”, “perfusion improving amount” or “pharmaceutically effective amount” refer to a therapeutic dose or regimen that result in a therapeutic or beneficial effect following its administration to a subject. The infarct area-improving, infarct area-improving, perfusion-improving, therapeutic, or pharmaceutical effect may be curing, minimizing, preventing or ameliorating a disease or disorder, or may have any other infarct area-improving, infarct area-improving, perfusion-improving, therapeutic, or pharmaceutical beneficial effect. The effective amount of the composition may vary with the age and physical condition of the biological subject being treated, the severity of the condition, the duration of the treatment, the nature of concurrent therapy, the timing of the infusion, the specific compound, composition or other active ingredient employed, the particular carrier utilized, and like factors.

Examples of formats for such embodiments may include, without limitation, MEMRI or MEMRI-DEMRI analysis at two or more time points, where a first time point is a diagnosed but untreated patient; and a second or additional time point(s) is a patient treated with a candidate therapeutic agent or regimen. An additional time point may include a patient treated with a candidate therapeutic agent or regimen, and challenged for the disease, for example a cardiac stress test.

In such clinical trial formats, each set of time points may correspond to a single patient, to a patient group, e.g. a cohort group, or to a mixture of individual and group data. Additional control data may also be included in such clinical trial formats, e.g. a placebo group, a disease-free group, and the like, as are known in the art. Formats of interest include crossover studies, randomized, double-blind, placebo-controlled, parallel group trial is also capable of testing drug efficacy, and the like. See, for example, Clinical Trials: A Methodologic Perspective Second Edition, S. Piantadosi, Wiley-Interscience; 2005, ISBN-13: 978-0471727811; and Design and Analysis of Clinical Trials: Concepts and Methodologies, S. Chow and J. Liu, Wiley-Interscience; 2003; ISBN-13: 978-0471249856, each herein specifically incorporated by reference.

In one embodiment, a blinded crossover clinical trial format is utilized. A patient alternates for a set period of time, e.g. one week, two weeks, three weeks, or from around about 7-14 days, or around about 10 days, between a test drug and placebo, with a 4-8 week washout period. In another embodiment a randomized, double-blind, placebo-controlled, parallel group trial is used to test drug efficacy.

In all such methods, the MEMRI or MEMRI-DEMRI analysis is performed at multiple time points, with a setting and administration protocol that permits the detection of intermediate zone cells in the PIR.

### Databases of Analyses

Also provided are databases of MRI analyses. Such databases will typically comprise analysis profiles of various individuals following a clinical protocol of interest etc., where such profiles are further described below.

The profiles and databases thereof may be provided in a variety of media to facilitate their use. “Media” refers to a manufacture that contains the expression profile information of the present invention. The databases of the present invention can be recorded on computer readable media, e.g. any medium that can be read and accessed directly by a computer. Such media include, but are not limited to: magnetic storage media, such as floppy discs, hard disc storage medium, and magnetic tape; optical storage media such as CD-ROM; electrical storage media such as RAM and ROM; and hybrids of these categories such as magnetic/optical storage media. One of skill in the art can readily appreciate how any of the presently known computer readable mediums can be used to create a manufacture comprising a recording of the present database information. “Recorded” refers to a process for storing information on computer readable medium, using any such methods as known in the art. Any convenient data storage structure may be chosen, based on the means used to access the stored information. A variety of data processor programs and formats can be used for storage, e.g. word processing text file, database format, etc.

As used herein, “a computer-based system” refers to the hardware means, software means, and data storage means used to analyze the information of the present invention. The minimum hardware of the computer-based systems of the present invention comprises a central processing unit (CPU), input means, output means, and data storage means. A skilled artisan can readily appreciate that any one of the currently available computer-based system are suitable for use in the present invention. The data storage means may comprise any manufacture comprising a recording of the present information as described above, or a memory access means that can access such a manufacture.

A variety of structural formats for the input and output means can be used to input and output the information in the computer-based systems of the present invention. Such presentation provides a skilled artisan with a ranking of similarities and identifies the degree of similarity contained in the test expression profile.

### Additional Analysis

In combination with the methods of the invention, other methods of cardiovascular analysis may be used, including various well-known imaging techniques such as scintigraphy, myocardial perfusion imaging, gated cardiac blood-pool imaging, first-pass ventriculography, right-to-left shunt detection, positron emission tomography, single photon emission computed tomography, harmonic phase magnetic resonance imaging, echocardiography, and myocardial perfusion reserve imaging.

Where a range of values is provided, it is understood that each intervening value, to the tenth of the unit of the lower limit unless the context clearly dictates otherwise, between the upper and lower limit of that range and any other stated or intervening value in that stated range is encompassed within the invention. The upper and lower limits of these smaller ranges which may independently be included in the smaller ranges also is encompassed within the invention, subject to any specifically excluded limit in the stated range. Where the stated range includes one or both of the limits, ranges excluding either both of those included limits are also included in the invention.

Unless defined otherwise, all technical and scientific terms used herein have the same meaning as commonly understood by one of ordinary skill in the art to which this invention belongs. Although any methods and materials similar or equivalent to those described herein can also be used in the practice or testing of the described invention, the preferred methods and materials are now described. All publications mentioned herein are incorporated herein by reference to disclose and describe the methods and/or materials in connection with which the publications are cited.

As used herein and in the appended claims, the singular forms “a”, “and”, and “the” include plural referents unless the context clearly dictates otherwise. All technical and scientific terms used herein have the same meaning.

Each of the references cited herein is incorporated herein by reference in its entirety. The publications discussed herein are provided solely for their disclosure prior to the filing date of the present application. Nothing herein is to be construed as an admission that the described invention is not entitled to antedate such publication by virtue of prior invention. Further, the dates of publication provided may be different from the actual publication dates which may need to be confirmed independently.

The following examples are put forth so as to provide those of ordinary skill in the art with a complete disclosure and description of how to make and use the described invention, and are not intended to limit the scope of what the inventors regard as their invention nor are they intended to represent that the experiments below are all or the only experiments performed. Efforts have been made to ensure accuracy with respect to numbers used (e.g. amounts, temperature, etc.) but some experimental errors and deviations should be accounted for. Unless indicated otherwise, parts are parts by weight, molecular weight is weight average molecular weight, temperature is in degrees Centigrade, and pressure is at or near atmospheric.

### EXPERIMENTAL

### EXAMPLE 1

Our first clinical study demonstrated that the peri-infarct (PIR) ischemia detected by cardiac MRI (CMR) predicted future cardiovascular events (CVE) in this patient population. Our second clinical study reported that the scar non-transmurality and volume/percentage of scar predicted future CVE. Others have also shown that PIR lead to gradual deterioration of cardiac function and pathological left ventricular (LV) remodeling with ongoing apoptosis and collagen deposition. Our third clinical study confirmed that tissue heterogeneity in the non-transmural PIR predicted CVE. Other studies have also documented that the clinical importance of tissue heterogeneity in the PIR is independent of the actual infarct size. These studies confirmed that precise tissue characterization of the PIR was a significant predictor of future CVE while other traditional measurements, including scar presence, LV ejection fraction (LVEF), and LV volumes did not demonstrate any significant relationship with the incidence of CVE. Additionally, the tissue heterogeneity in the PIR of ICM has been recognized as an important substrate to trigger ventricular arrhythmias and induce pathological LV remodeling. Studies have confirmed that the heterogeneous PIR contains the critical isthmus for ventricular tachycardia and ablation therapy of PIR eliminates the arrhythmogenicity. Indeed, an effective diagnosis of PIR represents a critical unmet need in cardiovascular medicine.

Limitations of delayed-enhanced MRI (DEMRI). The current gold standard for myocardial viability is delayed-enhanced MRI (DEMRI), which predicts functional recovery and improved survival in ICM patients after revascularization. This technique exploits the T1-shortening effect of gadolinium (Gd), which distributes primarily within the extracellular space to generate a bright MRI signal in acutely or chronically infarcted myocardium. However, DEMRI does not provide direct cell viability information due to its nonspecific distribution properties. Despite the predictive capability of DEMRI, several groups have reported that DEMRI decreases significantly over time. Others have reported that DEMRI may overestimate areas of nonviable infarct by as much as 15% with the majority of the discrepancy lying within the heterogeneous PIR. Indeed, DEMRI may also be positive in regions of myocardial edema and inflammation, which may cause transient, reversible cardiac injury patterns. To date, there is limited ability to detect viable and, potentially, salvageable cardiomyocytes within the PIR of DEMRI territory. No established imaging strategy identifies these salvageable areas, which could have a meaningful survival impact for the patients. An alternative approach utilizing additional contrast agents to complement Gd may be necessary .

An alternative approach to evaluate myocardial viability is manganese-enhanced MRI (MEMRI). A biologically active contrast agent, manganese (Mn²⁺), specific to the viable cardiomyocytes will complement the anatomical DEMRI data of the injured myocardium. Mn²⁺ is a divalent cation that enters cells via voltage-gated calcium channels. MEMRI exploits the T1 shortening effect of Mn²⁺ and limits the uptake to viable, metabolically active cells. The intracellular accumulation of Mn²⁺ increases positive MRI signal intensity due to a T1-shortening effect, targeting the viable cardiomyocytes robustly. Mn²⁺ is a naturally occurring essential micronutrient and an antioxidant excreted via the hepatobiliary system. Recent MnDPPP chelation and Ca²⁺ supplementation have enabled clinical applications by eliminating cardiac and neurologic toxicity. MEMRI improves the characterization of the injured myocardium and allows close correlation with histopathology. The resulting pattern of intracellular enhancement allows direct, high-resolution, steady-state imaging of myocardial viability. Its uptake is dependent upon the metabolic function of the viable cells.

Although Mn²⁺ was recognized as the first MRI contrast agent, toxicity hindered widespread use. However, the use of non-chelated Mn²⁺ (12%) with Ca²⁺ supplementation (calcium gluconate 10%) in EVP1001 has enabled clinical application by eliminating toxicity. With 5 unpaired electrons, Mn²⁺ is among the most effective of all potential metal ion-based MR contrast agents. Additionally, Mn²⁺ is a naturally occurring essential micronutrient, a natural antioxidant, and excreted via hepatobiliary system. Used widely in neuronal imaging, the uptake of Mn²⁺ into viable myocardial cells has also been well-documented. Recent MEMRI studies have demonstrated improved tissue characterization of the infarcted myocardium through direct visualization of the viable myocardium and found close correlation with histopathology of the infarct volumes.

Active intracellular accumulation of Mn²⁺ generates high signal from the viable cardiac cells in normal region, no signal from the non-viable cells in the intra-infarct region (IIR), and intermediate signal from the viable but injured cells in the peri-infarct region (PIR). The resulting pattern of intracellular enhancement allows high-resolution, steady-state imaging of myocardial viability and injury.

In order to delineate the PIR, dual contrast MEMRI and delayed enhanced MRI (MEMRI-DEMRI) strategy has been developed to detect the viable myocardium within the PIR where there are both positive DEMRI scar (non-viable) and positive MEMRI signal (viable). This heterogeneity indicates that a significant population of cardiomyocytes may be alive with intact Ca²⁺-channel function (MEMRI positive) in this region despite large amounts of surrounding necrotic tissue (DEMRI positive). Indeed, our tissue electron microscopy analysis of these overlapping zones of MEMRI and DEMRI signal demonstrate morphologically and ultrastructurally intact cardiomyocytes within the DEMRI-positive zones, corroborating the presence of viable and salvageable cardiomyocytes in the PIR. These injured but viable cardiomyocytes may trigger ventricular arrhythmia and/or LV remodeling in HF. A definitive study to evaluate how cell therapy alters these pockets of viable tissue directly will impact the clinical strategies for this critical unmet clinical need in cardiovascular medicine.

Mechanism and Safety Profile of MEMRI Contrast Agent. The ability of MEMRI to detect the viable myocardium directly allows precise evaluation of the progressive changes in the peri-infarct region. The mechanism of MEMRI contrast agent, developed in collaboration with Eagle Vision Pharmaceutical Inc., SeeMore™ (EVP1001), is based on an active ingredient consisting of non-chelated Mn²⁺ (12%) with calcium gluconate (10%). The non-chelated property of Mn²⁺ enables safe and rapid uptake by the Ca²⁺-channels of the viable cells, allowing highly effective intracellular targeting of viable cardiac cells with high level of safety and unique magnetic properties. The Ca²⁺ provides for safety, avoiding the cardiac depression that occurs with intravascular Mn²⁺ alone and does not alter the fundamental distribution or enhancement properties of Mn²⁺. This combination of Mn²⁺ with Ca²⁺ provides the unique intracellular enhancing features of Mn²⁺ (that otherwise requires radioactive tracers) while avoiding cardiac suppression or other untoward effects.

The chemical property of Mn²⁺ allows the active ingredient to traverse readily into the extra-cellular and -vascular space; however, Mn²⁺ does not enhance these regions without the presence of viable cells. It is actively taken up by live, active cells and binds to intracellular components such as the mitochondria to provide significant intracellular enhancement. If there are no live, active cells, there will be little to no enhancement by manganese, unlike other available magnetic imaging agents which are nonspecific, extracellular enhancing agents. For an example, gadolinium (Gd) does not get taken up into the cells. Instead, the infarct or edematous regions have an abnormally high “extracellular fluid” space relative to normal tissue and, therefore, show nonspecific/extracellular enhancement. MEMRI does not enhance such conditions because its enhancement relies on the specific, active uptake into live cells.

The published data on EVP have demonstrated high dose safety studies aimed at eliciting signs of toxicity at target organs. The safety index of 45 to 90 corresponded more favorably than the safety index of widely used clinical imaging agents (10 to 20 for iodinated X-ray products and 20 to 60 for gadolinium chelate MRI products). The human dose escalation study demonstrated safety, tolerance, pharmacokinetic (PK) profile, and multiple clinical parameters, which demonstrated safety at all doses tested. Phase I and II clinical trials have been completed and demonstrated no significant safety concerns. This specific, active intracellular uptake is one of the key attributes that offers potential benefit of cellular MRI of the myocardium. The labeling efficiency of Mn²⁺ in single cell culture of human bone marrow stem cells and human embryonic stem cells was studied and published.

Basic MEMRI parameters, including T₁ and T₂ values, were measured and validated in different concentration of Mn²⁺ (0.01-3.0 mM). The data were analyzed to extract T₁ and T₂ values through nonlinear least-square fits to the inversion recovery and spin-echo decay curves, respectively. The MEMRI viability signal was validated by transducing the cells with luciferase RG to detect BLI signal. MEMRI signal was modulated by the Ca²⁺ channel activity using a calcium-channel antagonist (verapamil) and agonist ((s)-Bay K8644), demonstrating subsequent decreased and increased MEMRI signal, respectively. These unique in vivo properties allow the infarcted to take up negligible amount, peri-infarct regions reduced amount, and normal tissue greatest amount for MEMRI enhancement. These properties enable precise determination of the baseline cardiac injury and subsequent restoration following stem cell therapy by delineating the direct changes in the regional viability at a cellular level. An FDA IND approval was obtained by the PI and a pilot clinical trial of 6 ischemic cardiomyopathy patients has demonstrated clear delineation of peri-infarct injury.

Dual contrast MEMRI-DEMRI. Transmural DEMRI-positive myocardial regions would traditionally indicate non-viable scar. However, at-risk myocardium that falls outside the infarct region and inside the PIR may contain viable cells. The novel dual contrast MEMRI-DEMRI highlights the effective of the Mn contrast agent, pointing to the viable cardiomyocytes within the PIR of transmural delayed enhancement. The discrepancy lies within the PIR that are positive for both MEMRI (viable) and DEMRI (non-viable) signal. The PIRs also display lower SNR by MEMRI than remote zones and lower SNR by DEMRI than core infarct zones, reflecting the heterogeneity of PIR with significant population of viable cardiomyocytes with intact Ca²⁺-channel function (MEMRI positive) in the PIR of the adjacent necrotic tissue (DEMRI positive).

Our histopathological analysis of these overlapping zones of MEMRI and DEMRI signal demonstrate morphologically and ultrastructurally intact but injured cardiomyocytes within DEMRI-positive zone consistent with previous publications regarding DEMRI overestimation of scar volume. Clinically, there is a critical need to detect truly viable myocardium at various time points post-MI to assist decisions on revascularization. Despite early-invasive revascularization, poorer outcomes have been demonstrated in certain subsets of patients, prompting recent changes in revascularization guideline. Thus, the patients in the peri-MI period may greatly benefit from accurate imaging of at-risk, viable myocardium that could be jeopardized.

MEMRI-DEMRI approach addresses an unmet need to predict the overall propensity of PIR to develop ventricular arrhythmia and/or remodeling. More importantly, the effects of cell, revascularization, and/or medical therapies on PIR can be delineated through the detection of progressive changes in regional viability and tissue heterogeneity.

Peri-infarct region (PIR) in ischemic cardiomyopathy. Ischemic cardiomyopathy (ICM) is the primary etiology of advanced heart failure (HF), the leading diagnosis of hospital admissions in the US. Clinical studies have confirmed that the high morbidity and mortality of HF are associated with ventricular arrhythmias and LV remodeling in the peri-infarct region (PIR). Indeed, patients with a history of acute MI and LV dysfunction have a 6 month mortality >10%, one third of which is attributed to sudden cardiac death. The critical role of tissue heterogeneity in the PIR, independent of actual infarct size, has been recognized as an important substrate to trigger these ventricular arrhythmias.

Preclinical studies have confirmed that the heterogeneous PIR contains the critical isthmus for ventricular tachycardia and successful therapy requires ablation of the isthmus. Studies have also demonstrated that revascularization of the ischemic PIR results in a lower incidence of ventricular arrhythmias and LV dilatation. While revascularization may mitigate ventricular arrhythmias acutely, the Multicenter UnSustained Tachycardia Trial (MUSTT) noted that following a reperfused MI, patients were increasingly likely to have ventricular arrhythmias as their infarct matured. Therefore, an accurate tissue characterization of the PIR is critical in determining which patients will benefit from revascularization and/or medical therapy targeted at PIR. Precise tissue characterization of the PIR predicts future cardiovascular events while traditional measures including scar presence, LVEF, and LV volumes did not demonstrate such significance.

### EXAMPLE 2

Dual contrast enhanced cardiac MRI using manganese and gadolinium in patients with severe ischemic cardiomyopathy detects the peri-infarct region (PIR)

Delayed Enhanced MRI (DEMRI) with gadolinium (Gd) is used as gold standard for diagnosis of myocardial infarction. However, the non-specific property of Gd overestimates the infarct size. Conversely, manganese (Mn2+) enters only the live, active cardiomyocytes via L-type Ca2+ channels. From our earlier work in animal MI models, manganese-enhanced MRI (MEMRI) has demonstrated its utility in identifying the viable, nonviable, and injured myocardium. We performed the “first in human” dual-contrast MEMRI-DEMRI to assess the efficacy of MEMRI-DEMRI to identify the periinfarct region (PIR) in patients with severe ischemic cardiomyopathy (ICM).

Methods

5 ICM patients (Class I-Ill CHF) have been enrolled (5 male, mean age 60±7 years). Cardiac MRI was performed using a 3.0T MRI scanner (Signa 3T HDx, GE HealthCare, USA) with an 8 channel cardiac coil (3.0T HD Cardiac Array, GE HealthCare, USA). LV functional images and DEMRI were acquired on the first day of this study, and MEMRI was acquired on the following week. (1) LV function: SSFP, flip angle (FA) 45, slice thickness (ST) 8.0 mm, matrix 224×224, FOV 35.0 cm; (2) DEMRI: FGRE-IR, TR 6.0, TE 2.8, TI 200-300, FA 15, ST 8.0 mm, matrix 224×192, FOV 35.0 cm, 0.2 mmol/kg Gd (Magnevist, Bayer HealthCare, Germany); and (3) MEMRI: FGRE-IR, TR 6.0, TE 2.8, TI 600-700, FA 15, ST 8.0 mm, matrix 224×192, FOV 35.0 cm, 1 mmol/kg Mn contrast agent, as described in U.S. Pat. No. 5,980,863, were performed. The infarct volumes were determined as 3 standard deviations (SDs) above mean on DEMRI and 2 SDs below mean on MEMRI using a Cardiac MRI Software (CMR42, Circle Cardiovascular Imaging Inc., Canada).

### Results

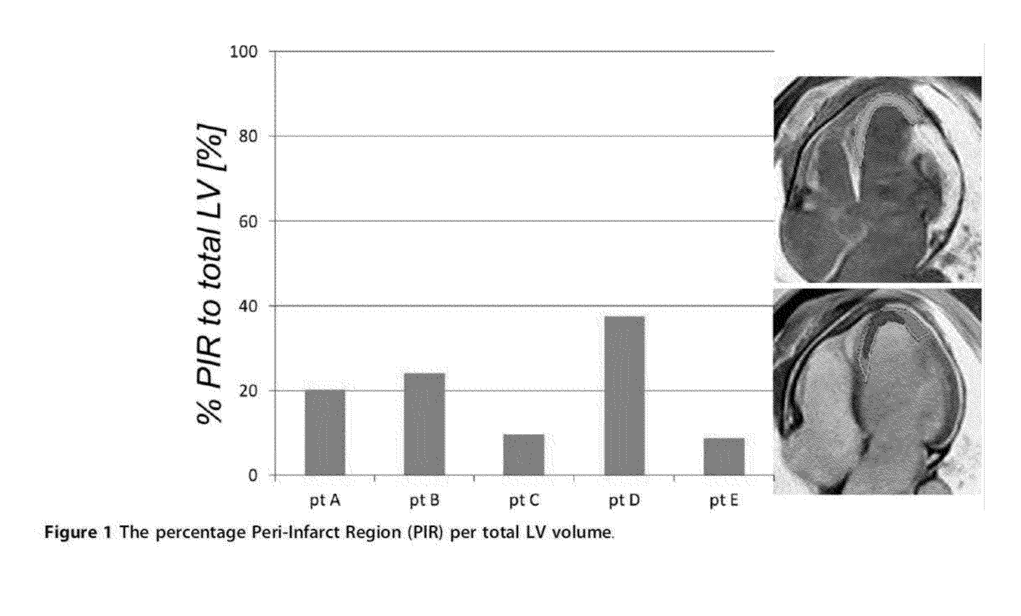

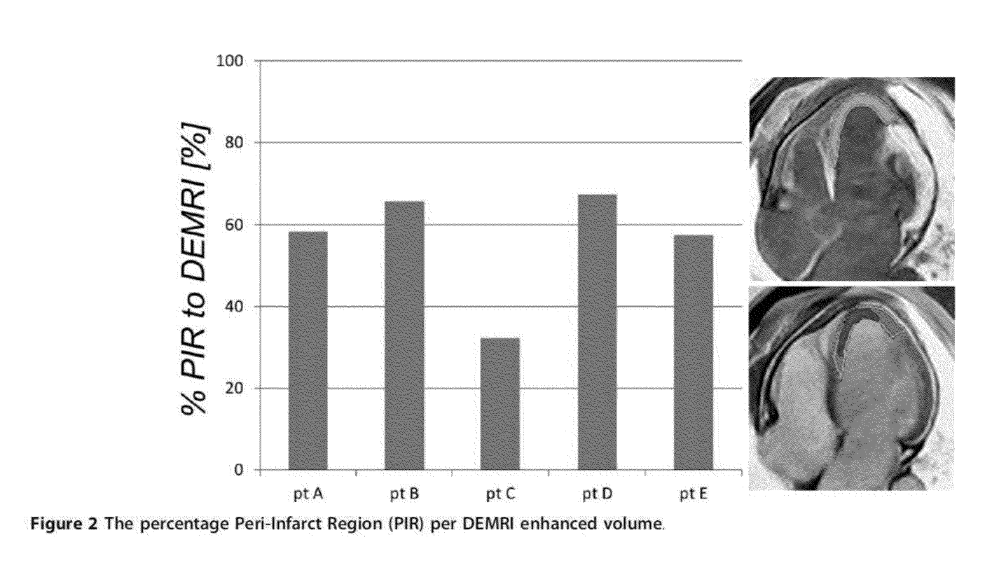

The average LVEF was 35±4%. The % enhanced DEMRI infarct volume (34±11%*) was significantly (*p<0.05) higher than the % defect MEMRI infarct volume (14±3%). The PIR was calculated as the difference between DEMRI and MERMRI. The mean % PIR per total LV and the mean % PIR per DEMRI enhancement were 20±12% (FIGS. 1) and 56±16% (FIG. 2), respectively.

Conclusions

The non-viable myocardium volume, appearing as MEMRI defect, was significantly smaller than the DEMRI enhancement. The discrepancy between DEMRI and MEMRI represents the PIR or “area-at-risk”. Therefore, our results show that the dual MEMRI-DEMRI contrast can clearly delineate the PIR by integrating the biology of viable myocardium and anatomy of non-viable myocardium. This dual contrast approach delineates the area-at-risk and predicts clinical outcomes from revascularization.

We claim:

1. A method for analysis of heterogeneous tissue, the method comprising performing manganese enhanced MRI on the tissue, and determining the presence of zone of tissue where intracellular accumulation of Mn²⁺ generates high signal from the viable cells; no signal from the non-viable cells; and intermediate signal from viable but injured cells.

2. The method of claim 1, wherein the heterogeneous tissue comprises a myocardial infarct and peri-infarct region.

3. The method of claim 1, wherein the heterogenous tissue is atrial wall tissue.

4. The method of claim 1, wherein imaging is performed with a contrast agent comprising non-chelated Mn²⁺ and calcium gluconate.

5. The method of claim 1, wherein the analysis further comprises a sequential or concomitant analysis by delayed-enhanced MRI (DEMRI) that distributes primarily within the extracellular space to generate a bright MRI signal in infarcted tissue.

6. The method of claim 5, wherein the difference between the infarct size obtained with DEMRI is subtracted from the infarct size obtained with MEMRI to provide a measurement of viable cells in the heterogeneous tissue.

7. The method of claim 1, wherein the analysis is performed for at least 2 time points.

8. The method of claim 7, wherein therapeutic intervention is performed between the two time points.

9. The method of claim 8, wherein the therapeutic intervention is performed in the context of a clinical trial.
